(19) United States
(12) Patent Application Publication
(10) Pub. No.: US 2002/0004592 A1
(43) Pub. Date: Jan. 10, 2002

(54) Method for amplifying full length single strand polynucleotide sequences

(76) Inventors: James R. Connor, Hershey, PA (US); Zheng Ye, Montreal (CA)

Correspondence Address: ZARLEY, MCKEE, THOMTE, VOORHEES & SEASE, ATTN: PENNSYLVANIA STATE UNIVERSITY, 801 GRAND AVENUE, SUITE 3200, DES MOINES, IA, 50309-2721, US

(21) Appl. No.: 09/780,762

(22) Filed: Feb. 9, 2001

Related U.S. Application Data
(60) Provisional application No. 60/181,615, filed on Feb. 10, 2000.
(60) Provisional application No. 60/203,035, filed on May 9, 2000.

(51) Int. Cl. C12P 19/34; C07H 21/04

(52) U.S. Cl. 536/24.3; 435/91.1

(57) ABSTRACT

Applicants have identified a novel amplification method that uses two specific primers to clone both the 5′ and 3′ cDNA ends in a single reaction. This new method also uses the first strand of cDNA obtained after reverse transcription of mRNA rather than double stranded cDNA, further increasing efficiency of amplification. According to the invention the first strand of cDNA is self-ligated using a DNA ligase.

### CROSS REFERENCE TO RELATED APPLICATIONS

This application is a continuation of provisional application Ser. No. 60/181,615 filed Feb. 10, 2000, priority is claimed under 35 U.S.C. § 120. This application is also claiming priority to provisional application Ser. No. 60/203,035 filed May 9, 2000.

### BACKGROUND OF THE INVENTION

Molecular cloning has enabled the study of the structure of individual genes of living organisms. The method traditionally required the replication of genetic sequences of plasmids or other vectors during cell division. Perhaps the most significant advancement in molecular cloning was the development of a DNA amplification procedure based on an in vitro rather than in vivo process, known as the polymerase chain reaction (PCR). This method produces large amounts of a specific DNA fragment from a complex DNA template in a simple enzymatic reaction. Cell-free gene amplification by PCR has simplified many of the standard procedures for cloning, sequencing, analyzing and ultimately modifying nucleic acids. The method utilizes a DNA polymerase and two oligonucleotide primers to synthesize a specific DNA fragment from a template sequence.

The amount of starting material needed for PCR can be as little as a single molecule rather than the usual millions of molecules required for standard cloning and molecular biological analysis. Although purified DNA is used in many applications, it is not required for PCR, and crude cell lysates also provide excellent templates. The DNA need not even be intact, in contrast to the requirements of other standard molecular biological procedures, as long as some molecules exist that contain sequences complementary to both primers. The speed and sensitivity of PCR have been widely recognized by scientists in both medicine and basic biology, and the method has been applied to problems that a few years ago were thought to be inaccessible to molecular analysis.

The basic method has been refined and optimized to even further increase the speed and accuracy of amplification. One problematic area of PCR involves the amplification identification of the 5′ and 3′ ends of a sequence, since PCR only amplifies from primer to primer, regions outside of the primer area cannot be amplified by regular PCR. A number of methods have been developed to try to clone cDNA ends by using PCR technique including RACE, anchored or single-sided PCR, inverse PCR, ligation-anchored PCR and RNA ligase-mediated RACE.

The RACE method uses one specific primer coupled a non-specific primer. Thus, because the non-specific primer could interact with any mRNA this method tends to generate numerous false positives resulting in decreased efficiency. Despite improvements in the RACE procedure, several limitations remain. Usually, the 5′-ends mapped by techniques based on homopolymer tailing or oligonucleotide ligation of the double strand cDNA do not correspond to the actual transcription start sites since premature termination of the reverse transcriptase results in size heterogeneity of the RACE products and the shortest or most abundant DNA products are preferentially amplified. Approaches which involve ligation of oligonucleotides to the 5′-ends of the mRNA before cDNA synthesis have often proved to be technically difficult and, as with all anchored or single-sided PCR methods, generate non-specific product due to use of the anchor primer. Finally, important information on tissue-specific changes in the 5′-ends of mRNAs which arise from alternative splicing and promoter usage is not readily obtained from the existing RACE methods.

Despite the availability of numerous approaches for cloning cDNA, it remains an arduous task, particularly when it is necessary to obtain a complete sequence or when attempting to clone a rare sequence.

As can be seen there is a need in the art for a method of cloning nucleotide sequences that can specifically amplify the 5′ and 3′ ends of the molecule in a single reaction.

It is an object of the present invention to provide a method for amplifying cDNA by PCR that is rapid and specifically includes the 3′ and 5′ ends of cDNA.

It is an object of the present invention to provide a method for amplifying cDNA by provide circularized first strand cDNA as template.

It is yet another object of the invention to provide a cloning method that can amplify 3′ and 5′ ends of cDNA in a single reaction.

It is yet another object of the invention to provide a cloning method that is more specific and enables more accurate characterization of genes.

It is yet another object to provide a cloning method with increased specificity by two gene specific primers.

These and other objects of the invention will become apparent from the detailed description of the invention which follows.

### BRIEF SUMMARY OF THE INVENTION

Applicants have identified a novel amplification method that uses two specific primers to clone both the 5′ and 3′ polynucleotide ends in a single reaction. This new method also uses a single strand of polynucleotide, and can be used to amplify the first single cDNA strand obtained after reverse transcription of mRNA rather than double stranded cDNA, further increasing accuracy and efficiency of amplification. According to the invention the single strand of polynucleotide is self-ligated to form a circular structure. Two gene specific primers designed from known target sequences within the polynucleotide are introduced to amplify the 5′ and 3′ ends. Design of these primers is critical as each primer will have a 3′ end towards one of the polynucleotide ends. PCR or another primer extension amplification procedure is then used to amplify the resulting specific nucleotide sequences. The resulting amplified product will include the desired 3′ and 5′ ends of cDNA outside of the two primers. This product can then be used for a number of molecular biology protocols including diagnostics, sequencing, or mutation.

In a preferred embodiment the amplified polynucleotide is sequenced. To sequence the polynucleotide, the amplified product may then be inserted into a plasmid vector for sequencing. Based on sequence information, new primers may then be designed to clone the full-length cDNA, of a particular gene.

According to the invention, human glyceraldehyde-3-phosphate dehydrogenase (GAPDH) cDNA, NEMO cDNA, Thy-1 cDNA and one iron inhibited ABC transporter cDNA were cloned in full length using this approach. Compared to records in GenBank, applicants approach resulted in longer sequences that are consistent with the genomic DNA sequence data.

The following terms as used herein shall be defined as follows. Units, prefixes, and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range and include each integer within the defined range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. Unless otherwise provided for, software, electrical, and electronics terms as used herein are as defined in The New IEEE Standard Dictionary of Electrical and Electronics Terms (5^{th} edition, 1993). The terms defined below are more fully defined by reference to the specification as a whole.

By “amplified” is meant the construction of multiple copies of a nucleic acid sequence or multiple copies complementary to the nucleic acid sequence using at least one of the nucleic acid sequences as a template. Amplification systems often herein refer to the polymerase chain reaction (PCR) system, however the invention is not so limited and is intended to include ligase chain reaction (LCR) system, nucleic acid sequence based amplification (NASBA, Canteen, Mississauga, Ontario), Q-Beta Replicase systems, transcription-based amplification system (TAS), and strand displacement amplification (SDA). See, e.g., Diagnostic Molecular Microbiology: Principles and Applications, D. H. Persing et al., Ed., American Society for Microbiology, Washington, D.C. (1993). The product of amplification is termed an amplicon.

The term “hybridization complex” includes reference to a duplex nucleic acid structure formed by two single-stranded nucleic acid sequences selectively hybridized with each other.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA).

The term “isolated” refers to material, such as a nucleic acid or a protein, which is: (1) substantially or essentially free from components that normally accompany or interact with it as found in its naturally occurring environment. The isolated material optionally comprises material not found with the material in its natural environment; or (2) if the material is in its natural environment, the material has been synthetically (non-naturally) altered by deliberate human intervention to a composition and/or placed at a location in the cell (e.g., genome or subcellular organelle) not native to a material found in that environment. The alteration to yield the synthetic material can be performed on the material within or removed from its natural state. For example, a naturally occurring nucleic acid becomes an isolated nucleic acid if it is altered, or if it is transcribed from DNA which has been altered, by means of human intervention performed within the cell from which it originates. See, e.g., Compounds and Methods for Site Directed Mutagenesis in Eukaryotic Cells, Kmiec, U.S. Pat. No. 5,565,350; In Vivo Homologous Sequence Targeting in Eukaryotic Cells; Zarling et al., PCT/US93/03868. Likewise, a naturally occurring nucleic acid (e.g., a promoter) becomes isolated if it is introduced by non-naturally occurring means to a locus of the genome not native to that nucleic acid. Nucleic acids which are “isolated” as defined herein, are also referred to as “heterologous” nucleic acids.

As used herein, “nucleic acid” includes reference to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

By “nucleic acid library” is meant a collection of isolated DNA or RNA molecules which comprise and substantially represent the entire transcribed fraction of a genome of a specified organism. Construction of exemplary nucleic acid libraries, such as genomic and cDNA libraries, is taught in standard molecular biology references such as Berger and Kimmel, Guide to Molecular Cloning Techniques, Methods in Enzymology, Vol. 152, Academic Press, Inc., San Diego, Calif. (Berger); Sambrook et al., Molecular Cloning—A Laboratory Manual, 2^{nd} ed., Vol. 1-3 (1989); and Current Protocols in Molecular Biology, F. M. Ausubel et al., Eds., Current Protocols, a joint venture between Greene Publishing Associates, Inc. and John Wiley & Sons, Inc. (1994).

As used herein, “polynucleotide” includes reference to a deoxyribopolynucleotide, ribopolynucleotide, or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons as “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including among other things, simple and complex cells.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. The terms “polypeptide”, “peptide” and “protein” are also inclusive of modifications including, but not limited to, glycosylation, lipid attachment, sulfation, gamma-carboxylation of glutamic acid residues, hydroxylation and ADP-ribosylation. It will be appreciated, as is well known and as noted above, that polypeptides are not entirely linear. For instance, polypeptides may be branched as a result of ubiquitination, and they may be circular, with or without branching, generally as a result of posttranslation events, including natural processing event and events brought about by human manipulation which do not occur naturally. Circular, branched and branched circular polypeptides may be synthesized by non-translation natural process and by entirely synthetic methods, as well. Further, this invention contemplates the use of both the methionine-containing and the methionine-less amino terminal variants of the protein of the invention.

As used herein, “vector” includes reference to a nucleic acid used in transfection of a host cell and into which can be inserted a polynucleotide. Vectors are often replicons. Expression vectors permit transcription of a nucleic acid inserted therein.

### BRIEF DESCRIPTION OF THE DRAWINGS

FIG. 1 is a schematic illustrating the principle of cDNA cloning by the methods of the invention. RNA reverse transcriptase without RNase H activity was used to synthesize the first strand cDNA. The mRNA template was degraded by RNases and the remaining first strand cDNA was purified and self-ligated to form circular molecules. Two gene specific primers (GSP 1 and GSP 2) were designed from a segment of known sequence.

FIG. 2A depicts the first PCR amplification to determine the size of the selected gene products visualized using ethidium bromide. The products were analyzed on 1% agarose gel. M1 and M2 are DNA molecular weight markers; 1, GAPDH; 2, NADH dehydrogenase 1 beta subcomplex 9; 3, DNA-binding Protein, TAXREB107; 4, NEMO Protein; 5, IRP-l; 6, calpain large polypeptide L2; 7, Thy-1; 8, iron-inhibited ABC transporter. The calculated sizes for GAPDH, NEMO were longer than that reported in GenBank and the size of the DNA binding protein TAXREB107 was similar to that reported in GenBank.

FIG. 2B depicts a second PCR amplification using new primers was performed on those genes whose size did not correspond to the size indicated by Northern blot analysis or to the size reported in GenBank. Lane 1, IRP-1; lane 2, calpain, large Polypeptide L2; lane 3, NADH dehydrogenase (ubiquinone) 1; lane 4, Thy-1; lane 5, iron-inhibited ABC transporter. Using the second set of primers, we obtained calculated lengths longer than that reported in GenBank for all five of the cDNAs examined. (M2 and M1 are DNA molecular weight markers.)

FIG. 3 depicts PCR amplification of cDNAs to confirm the novel cDNA sequences. The products of the PCR reaction were analyzed on 1% agarose gel. M is the DNA molecular weight Marker. Lane 1, GAPDH; lane 2, NEMO; lane 3, IRP-1; lane 4, calpain large polypeptide L2; lane 5, Thy-1; lane 6, ABC transporter (small band); lane 7, ABC transporter (large band). The sequences obtained by this amplification step correspond to the sequences obtained in the previous two PCR amplifications confirming that our cloning method is accurate.

### DETAILED DESCRIPTION OF THE INVENTION

According to the invention, a method for amplification of a polynucleotide which includes the amplification of 3′ and 5′ ends of the molecule in a single reaction is disclosed. According to the invention a single strand of polynucleotide, preferably DNA, and even more preferably cDNA may be used. The single strand polynucleotide is then self-ligated to form a circular nucleic acid structure. Essentially the 5′ and 3′ ends are joined together and thus become part of the amplification reaction product. This is accomplished by a DNA or RNA ligase. Ligases are commercially available and these molecules are widely used in the art of molecular biology. Examples of such ligases include T4 RNA ligase, T4 DNA ligase and E. Coli DNA ligase from Gibco BRL. The preferred and most widely available ligase is T4 DNA ligase which is commercially available from a number of sources including Panvera, Stratagene, and Boeringer Mannheim.

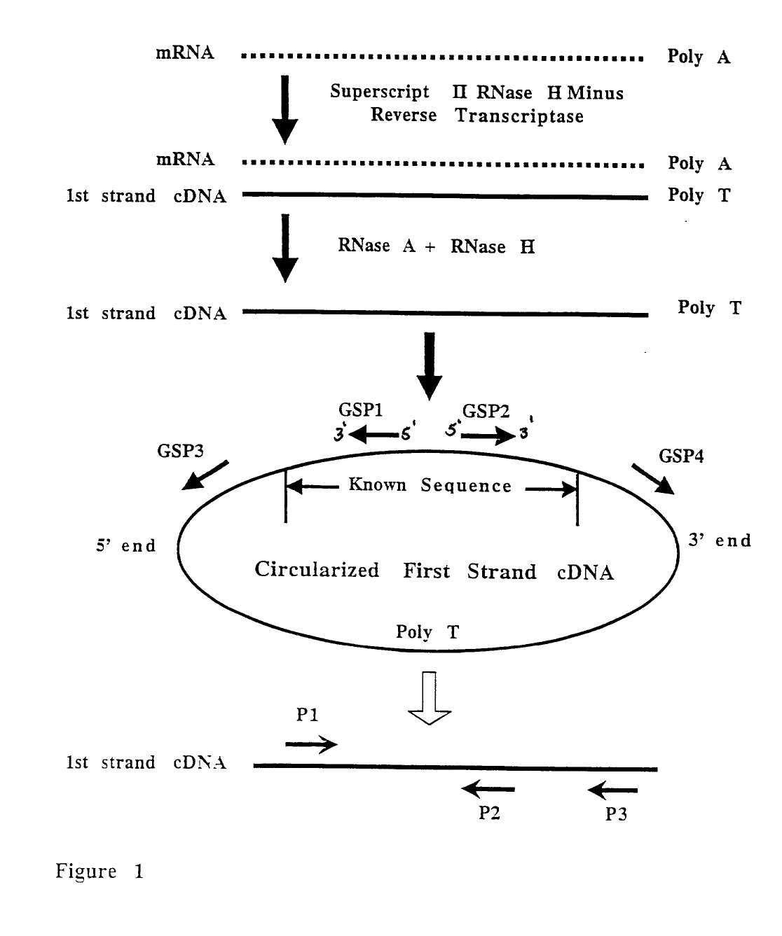

Once the circular nucleic acid is formed, then a template extension amplification reaction is carried out with gene specific primers. The design of the first and second primers differs from that of traditional PCR of cDNA first in that using a single nucleic acid strand as template. The primers are instead designed so that each one has a 3′ end of the primer which is toward either the 5′ or 3′ end of the polynucleotide. This means that the forward primer will typically be towards the 3′ end of the molecule and the reverse primer will be towards the 5′ end of the molecule. For example, if a known sequence comprises 5′-ATATATATGCGCGCGC-3′ a forward primer would be 5′-CGCGCGCG-3′ to hybridize with the 3′ end of the molecule and the second or reverse primer would be 5′-ATATATAT-3′ to hybridize with the 5′ end of the molecule and having its 3′ end towards the 5′ of the target gene. See FIG. 1. Design of primers for amplification and extension reactions are commonly known in the art of PCR amplification and the remainder of primer design is standard. A brief summary of oligonucleotide primer design is disclosed herein. In addition a discussion of primer design can be located in “Molecular biology Techniques Manual” third edition CRC Press, Editors, Coyne et al. available at www.uct.ac.za/microbiology/pcroptim.htm. In addition, there are a number of publically and commercially available computer programs to aid in design of primers including, BLAST, PrimerGen, Primer (Stanford), Amplify, Primer Design 1.04, PC-Rare, CODEHOP, Primer 3, and Net Primer (Premier Biosoft Int'l).

Typical background information in design of primers is as follows:

Primer Selection

Several variables must be taken into account when designing PCR Primers. Among the most critical are: primer length; melting temperature (Tₘ); specificity; complementary primer sequences; G/C content and polypyrimidine (T,C) or polypurine (A,G) stretches; 3′-end sequence. Each of these critical elements will be discussed in turn.

Primer Length

Since both specificity and the temperature and time of annealing are at least partly dependent on primer length, this parameter is critical for successful PCR. In general, oligonucleotides between 18 and 24 bases are extremely sequence specific, provided that the annealing temperature is optimal. Primer length is also proportional to annealing efficiency: in general, the longer the primer, the more inefficient the annealing. With fewer templates primed at each step, this can result in a significant decrease in amplified product. The primers should not be too short, however, unless the application specifically calls for it. As discussed below, the goal should be to design a primer with an annealing temperature of at least 50° C.

The relationship between annealing temperature and melting temperature is one of the “Black Boxes” of PCR. A general rule-of-thumb is to use an annealing temperature that is 5° C. lower than the melting temperature. Thus, when aiming for an annealing temperature of at least 50° C., this corresponds to a primer with a calculated melting temperature (Tₘ) ˜55° C. Often, the annealing temperature determined in this fashion will not be optimal and empirical experiments will have to be performed to determine the optimal temperature. This is most easily accomplished using a gradient thermal cycler like Eppendorf Scientific's Mastercycler® Gradient.

Melting Temperature (Tₘ)

It is important to keep in mind that there are two primers added to a PCR reaction. Both of the oligonucleotide primers should be designed such that they have similar melting temperatures. If primers are mismatched in terms of Tₘ, amplification will be less efficient or may not work at all since the primer with the higher Tₘ will mis-prime at lower temperatures and the primer with the lower Tₘ may not work at higher temperatures.

The melting temperatures of oligos are most accurately calculated using nearest neighbor thermodynamic calculations with the formula:

Tₘ^{primer}=(delta)H[(delta)S+R In(c/r)]−273.15° C.+16.6 log₁₀ [K+]

where H is the enthalpy and S is the entropy for helix formation, R is the molar gas constant and c is the concentration of primer. This is most easily accomplished using any of a number of primer design software packages on the market. (Sharrocks, A. D., The design of primers for PCR, in PCR Technology, Current Innovations, Griffin, H. G., and Griffin, A. M., Ed., CRC Press, London, 1994, 5-11). Fortunately, a good working approximation of this value (generally valid for oligos in the 18-24 base range) can be calculated using the formula:

Tₘ=2(AT)+4(GC).

The table below shows calculated values for primers of various lengths using this equation, which is known as the Wallace formula, and assuming a 50% GC content. (Suggs, S. V., et al., Using Purified Genes, in ICN-UCLA Symp. Developmental Biology, Vol. 23, Brown, D. D. Ed., Academic Press, New York, 1981, 683).

| Primer | Tₘ = 2 (AT) + | Primer | Tₘ = 2 (AT) + |
|---|---|---|---|
| Length | 4 (GC) | Length | 4 (GC) |
| 4 | 12° C. | 22 | 66° C. |
| 6 | 18° C. | 24 | 72° C. |
| 8 | 24° C. | 26 | 78° C. |
| 10 | 30° C. | 28 | 84° C. |
| 12 | 36° C. | 30 | 90° C. |
| 14 | 42° C. | 32 | 96° C. |
| 16 | 48° C. | 34 | 102° C. |
| 18 | 54° C. | 36 | 108° C. |
| 20 | 66° C. | 38 | 114° C. |

The temperatures calculated using Wallace's rule are inaccurate at the extremes of this chart.

In addition to calculating the melting temperatures of the primers, care must be taken to ensure that the melting temperature of the product is low enough to ensure 100% melting at 92° C. This parameter will help ensure a more efficient PCR, but is not always necessary for successful PCR. In general, products between 100-600 base pairs are efficiently amplified in many PCR reactions. If there is doubt, the product Tₘ can be calculated using the formula:

Tₘ=81.5+16.6 (log₁₀[K+]+0.41 (%G+C)−675/length.

Under standard PCR conditions of 50 mM KCL, this reduces to (Sharrocks, A. D., The design primers for PCR, in PCR Technology, Current Innovations, Griffin, H. G., and Griffin, A. M., Ed., CRC Press, London, 1994, 5-11).

Tₘ=59.9+0.41 (%G+C)−675/length

According to the invention, a primer extension amplification reaction is performed with the two sequence specific primers. This is preferably by PCR.

General Discussion of PCR Amplification of PCR Reaction

The polymerase chain reaction produces large amounts of a specific DNA fragment from a complex DNA template in a simple enzymatic reaction. The method utilizes a DNA polymerase and two oligonucleotide primers to synthesize a specific DNA fragment from a template sequence. Locally two small stretches of known unique sequence that flank the target are used to design two oligonucleotide primers. The length of the primers (usually from about 5 to about 30 bases) must be sufficient to overcome the statistical likelihood that their sequence would occur randomly in the overwhelmingly large number of nontarget DNA sequences in the sample. PCR is carried out in a series of cycles. Each cycle begins with a denaturation step to render the target nucleic acid single-stranded. This is followed by an annealing step during which the primers anneal to their complementary sequences so that their 3′ hydroxyl ends face the target. Finally each primer is extended through the target region by the action of DNA polymerase. These three-step cycles are repeated over and over until a sufficient amount of product is produced. A critical requirement is that the extension products of each primer extend far enough through the target region to include the sequences of the other flanking primer.

The earliest PCR experiments utilized the Klenow fragment of Escherichia coli DNA polymerase I at a temperature of 37° C. and often produced incompletely pure target product as judged by gel electrophoresis. The isolation of a heat-resistant DNA polymerase from Thermus aquaticus (Taq) allows primer annealing and extension to be carried out at an elevated temperature, thereby reducing mismatched annealing to nontarget sequences.

Another important advantage of Taq polymerase is that it escapes inactivation during each cycle, unlike the Klenow enzyme, which had to be added after every denaturation step. This has allowed automation of PCR using machines that have controlled heating and cooling capability. A number of thermocyclers are commercially available at relatively low cost.

PCR Specificity

Specificity is achieved by designing primers flanking the target that are of sufficient length so that their sequence is virtually unique in the genome. The specificity of the interaction of the primer with the desired template versus nontarget DNA is temperature and salt concentration dependent, and appropriate conditions must be determined empirically. The conditions of the reaction must also be compatible with full activity of the polymerase.

It is the usual practice to set up the reaction at room temperature and to begin it with a 92-96° C. denaturation step. It has been suggested that even while the samples are being prepared primer extension by the Taq DNA polymerase could occur. At room temperature there would be little specificity to primer-template interactions. Experiments have shown that some of the nonspecific amplification products can be eliminated under so-called “hot start” conditions. This approach keeps the sample at a temperature greater than the calculated annealing temperature for the specific primer before the reaction is started.

Details of the Reaction

In addition to a genomic DNA sample usually containing less than 1 (pmol) of specific target sequence, the 25-100 liter volume includes 20 nmol of each of the four deoxynucleoside triphosphates (dATP, dCTP, dGTP, and dTTP), 10 to 100 pmol of each primer, the appropriate salts and buffers and DNA polymerase. The nucleotide concentration must be sufficient to saturate the enzyme, but not so low or unbalanced as to promote misincorporation (see below). The primer concentration must be high enough to anneal rapidly to the single-stranded target and, in later stages of the reaction, faster than target-target reassociation. Temperature control and timing are also important. Denaturation must be efficient, but the temperature must not be too high or held for too long a period, because the Taq polymerase, although heat-resistant, is not indefinitely stable. The temperature used for annealing must maximize specific primer annealing and polymerase elongation but not sacrifice yield by reducing primer-template hybridization.

The reaction mixture is usually overlaid with mineral oil to prevent evaporation, thereby contributing to rapid thermal equilibration and eliminating a concentration of because the whole sample tube including the cap is heated, mineral oil is not required to prevent evaporation. In general, using 20-nucleotide-length primer sequences with a 50% GC content, denaturation at 92-96° C. for 30-60 seconds, annealing at 55-60° C. for 30 seconds, and extension at 72° C. for 1 minutes is satisfactory for targets less than 500 bp. It is often found that a simple two-step cycle (95° C. denaturation; 60° C. annealing and extension) also gives excellent results.

Properties of Thermostable Polymerase

The introduction of a thermostable DNA polymerase from Thermus aquaricus (Taq polymerase) into the PCR greatly simplified the PCR protocol and allowed the development of simple thermal cycling instruments to automate the reaction. It also dramatically increased the specificity and yield of the PCR by allowing primer annealing and extension to be carried out at higher temperatures. It has a temperature optimum of 75-80° C., depending on the DNA template. Under appropriate conditions, it is highly processive and has been reported to have the extension rate of >60 nucleotides per second at 70° C. using M13 phage DNA as template.

Recently, a variety of thermostable DNA polymerases with different properties have been isolated from other bacteria. One, from the thermoacidophilic archebacterium Sulfolobus acidocaldarius, has been shown to carry out polymerization at 100° C. (Klimczak, L. J., et al. 1985, Nucleic Acids Res. 13:5269-82; Elie, C., et al., 1988, Biochem. Biophys. Acta 951:261-67; Salhi, S., et al., 1989, J. Mol. Biol. 209:635-44), which could facilitate the amplification of regions of high secondary structure and enhance specificity. In the case of Taq polymerase, the enzymatic incorporation of modified bases such as 7-Aza dGTP has proved useful in the amplification of sequences with secondary structures in GC-rich regions (McConlogue, L., et al., 1988, Nucleic Acids Res. 16:9869). Some of the new thermostable polymerases may allow the efficient amplification of larger PCR products (E. Rose, personal communication). The introduction of thermostable accessory proteins may also prove helpful in increasing the processivity of polymerases during PCR and allow the amplification of longer products.

The search for new thermostable polymerases has resulted in the discovery of one with reverse transcriptase activity. (Myers, T. W., et al., 1991, Biochemistry 30:7661-66).

Finally, polymerases from Thermoplasma acidophilum, Thermococcus litoralis, and Methanobacterium thermoautotrophicum have been reported to have 3′-5′ exonuclease activities (Klimczak, L. J., et al., 1986, Biochemistry 25:4850-55; Hamal, A., et al., 1990, Eur. J. Biochem. 190:517-21; Cariello, N. F., et al., 1991, Nucleic Acids Res. 19:4193-98).

Amplified products according to the invention have a number of uses in molecular biology, examples of the same include typically any use for which PCR is currently used. These include but are not limited to the following:

A. Genome Mapping

Olson M., Hood L., Cantor C, Botstein D., “A common language for physical mapping of the human genome”, Science, Sep. 29, 1989, 245(4925):1434-5; Paabo, S., “Ancient DNA: Extraction, characterization, molecular cloning, and enzymatic amplification”, Proceedings of the National Academy of Sciences of the United States of America, 1989, v. 86, n.6.

B. Evolutionary Biology

Kocher T. D., Thomas W. K, Meyer A, Edwards S. V., Paabo S, Villablanca F. X, Wilson A. C., “Dynamics of mitochondrial DNA evolution in animals: amplification and sequencing with conserved primers”, Proceedings of the National Academy of Sciences of the United States of America, 1989 August, 86(16:6196-200); Paabo, S, Higuchi R. G, Wilson A. C., “Ancient DNA and the Polymerase Chain Reaction the Emerging Field of Molecular Archaeology”, Journal of Biological Chemistry, 1989.

C. Clinical Applications

Saiki R. K, Walsh P. S., Levenson C. H., Erlich H. A., “Genetic analysis of amplified DNA with immobilized sequence-specific oligonucleotide probes”, Proceedings of the National Academy of Sciences of the United States of America, 1989:6230-6234; White T. J, Madej R, Persing D. H., “The polymerase chain reaction: clinical applications”, Advances in Clinical Chemistry, 1992, 29:161-96; Leeflang E. P., Zhang L, Tavare S, Hubert R, Srinidhl J, MacDonald M. E., Myers R. H., DeYoung M, Wexler N. S., Gusella J. F., and others “Single sperm analysis of the trinucleotide repeats in the Huntington's disease gene: Quantification of the mutation frequency spectrum”, Human Molecular Genetics, 1995, v.4, n.9, 1519-1526.

D. Sequencing

Holland P. M., Abramson R. D., Watson R, Gelfand D. H., “Detection of specific polymerase chain reaction product by utilizing the 5′.(fwdarw).3′ exonuclease activity of Thermus aquaticus DNA polymerase”, Proceedings of the National Academy of Sciences of the United States of America, 1991, v.88, n.16; Higuchi R, Dollinger G, Walsh P. S., Griffith R., “Simultaneous amplification and detection of specific DNA sequences”, Biotechnology, 1992 April, 10(4).

E. Applying Unknown Sequence from Single Strand Template

Erlich H. A., Gelfand D. H., Saiki R. K., “Specific DNA Amplification”, Nature, Feb. 4, 1988, V.331, 461-462; Bugawan T. L., Saiki R. K., Levenson C. H., Watson R. W., Erlich H. A., “The Use of Non-Radioactive Oligonucleotide Probes to Analyze Enzymatically Amplified DNA for Prenatal Diagnosis and Forensic Hla Typing”, Bio-Technology, 1988; Kinzler W, Vogelstein G., “Whole genome PCR: application to the identification of sequences bound by gene regulatory proteins”, Nucleic Acids Research, May 25, 1989, 17(10):3645-53.

F. Amplifying Unknown Sequences from a Single Strand Template

Erlich H. A., Gelfand D. H., Saiki R. K., “Specific DNA Amplification”, Nature, Feb. 4, 1988, V.331, 461-462; Bugawan T. L., Saiki R. K., Levenson C. H., Watson R. W., Erlich H. A., “The Use of Non-Radioactive Oligonucleotide Probes to Analyze Enzymatically Amplified DNA for Prenatal Diagnosis and Forensic Hla Typing”, Bio-Technology, 1988; Kinzler W, Vogelstein B., “Whole genome PCR: application to the identification of sequences bound by gene regulatory proteins”, Nucleic Acids Research, May 25, 1989, 17(10):3645-53.

G. Altering Sequence

Scharf S. J, Horn G. T, Erlich H. A., “Direct cloning and sequence analysis of enzymatically amplified genomic sequences”, Science, Sep 5, 1986, 233(4768):1076-8; Saiki R. K., Bugawan T. L., Horn G. T., Mullis K. B., Erlich H. A., “Analysis of enzymatically amplified beta-globin and HLA-DQ alpha DNA with allele-specific oligonucleotide probes”, Nature, Nov 13-19, 1986, 324(6093):163-6; Saiki R. K., Gelfand D. H., Stoffel S, Scharf S. J., Higuchi R., Horn G. T., Mullis K. B., Erlich H. A., “Primer-directed enzymatic amplification of DNA with a thermostable DNA polymerase”, Science, Jan. 29, 1988, 239(4839):487-81; Erlich H. A., Gelfand, D. H., Saiki R. K., “Specific DNA Amplification”, Nature, Feb. 4, 1988, V. 331, 461-461; White T. J., Arnheim N., Erlich H. A., “The polymerase chain reaction”, Trends in Genetics, 1989 Jun, 5(6):185-9.

H. Sample Preparation

Arnheim N, Li H. H., Cui X. F., “PCR analysis of DNA sequences in single cells: single sperm gene mapping and genetic disease diagnosis”, Genomics, 1990 November, 8(3); Arnheim, N., White, T. J., Rainey, W. E., “Application of PCR: Organismal and Population Biology Polymerase Chain Reaction Can Produce Large Quantities of Specific DNA from Small Degraded and Impure Samples”, Bioscience, 1990, v. 40, n. 3, 194-182; Kellogg D. E., Sninsky J. J., Kwok, S., “Quantitation of HIV-1 proviral DNA relative to cellular DNA by the polymerase chain reaction”, Analytical Biochemistry, 1990, v. 189, n.2, 202-208.

In a preferred embodiment the methods of the invention are used to amplify a first strand CDNA from an mRNA sample obtained from cell or tissue or body fluids. In this embodiment the mRNA was transcribed by using reverse transcriptase without RNase H activity to form cDNA-RNA complex. The RNA is then degraded preferably by enzymes such as RNaseA and RNaseH. The resulting single strand of cDNA is then ligated to form a circularized strand by using a DNA ligase. Two gene specific primers, one directed toward the 5′ end and one directed toward the 3′ end, were used in touch-down PCR to amplify the specific cDNA ends. Preferably touchdown PCR is used. A cDNA band of correct size can be obtained on the first pass of this modification. If the correct size is not obtained on the first pass, amplification of cDNA ends can be repeated until the correct size of the cDNA is obtained.

This method was applied on eight mRNAs that had previously been shown to respond to cellular iron levels. According to the invention sequences were obtained for six mRNAs that were 43bp to 1324bp longer than that reported in GenBank and obtained the same length sequence for the other two mRNAs. Applicants amplification approach offers a more efficient method for cloning full-length cDNA and it may be used to replace the existing method of 5′ end cDNA extension. The particular advantage of this latter application is the ability to obtain untranslated regions of a cDNA that can provide information regarding the regulation of the gene.

Applicants invention provides the traditional methods of cloning full length cDNA include: hybridization screening of cDNA library and then amplification of mRNA 5′ end and 3′ end.

The necessity for preparation and screening of cDNA libraries has many disadvantages including: establishment of cDNA library is time consuming and expensive, screening cDNA library is also time consuming and hard to get the full sequence; very difficult to clone cDNA from rarely expressed mRNA.

The amplification of mRNA ends also has many disadvantages including: the background of PCR products is very high because the use of a non-specific primer in all of the amplification reaction as well as this primer bind to both ends of cDNA; low expressed mRNA cannot be cloned.

Both of these techniques are replaced by applicants invention which provides truly full length cDNA because the step of synthesizing second strand cDNA was saved by using the first strand cDNA as amplification template; direct ligation of 5′ end and 3′ end of first strand cDNA so that the amplification is performed by using two specific PCR primer; easy to perform because of the simple procedure and low expense. It is very easy to synthesize the first strand cDNA from specific source of mRNA and the specific primers can be synthesized from a small piece of known sequence.

The reagents suitable for applying the methods of the invention may be packaged into convenient kits. The kits provide the necessary materials, packaged into suitable containers. At a minimum, the kit contains a reagent that provides for self ligation of a polynucleotide such as a DNA or RNA ligase, a polymerase for an amplification reaction and a supply of four deoxyribonucleotide triphosphates (typically dATP, dGTP, dCTP, and dTTP).

The circularized cDNAs from different cell or tissue can be prepared in a kit that is ready to be used in PCR amplification of specific cDNA. It works just like a cDNA library but it is used for cloning specific cDNA by PCR, not for library screening.

Other reagents used for hybridization, prehybridization, DNA extraction, mRNA extraction, visualization, etc. may also be included, if desired.

The novel cloning method described in this application provides not only an alternative to existing methods but represents an improvement in the existing technology. The use of circularized cDNA for cloning is an advantage over existing methods because it minimizes the need to consider upstream and downstream relations in the cDNA template. Thus, two gene specific primers can be used in generating a sequence from unknown cDNA ends. Attempts to circularize double stranded cDNA as PCR templates were not successful because the background was unacceptably high (data not shown). In the development of this technique, we also found that T4 RNA ligase could not be used to form circularized cDNA molecules because the PCR reaction also produced a high background of nonspecific products when circularized single strand cDNAs was ligated by this strategy (data not shown).

Our results also show that this new method provides a powerful alternative to traditional cloning methods for obtaining full-length cDNA. Although most of the sequence data for the mRNAs we selected for analysis have been available from a number of entries of GenBank for a relatively long time and have undergone frequent updates, our results showed that their sequences were incomplete. The advantage of cloning full length cDNA with our method is that our approach overcomes two defects that may limit success in full length cloning.

The first problem applicants technique circumvents is the requirement to synthesize double stranded cDNA following reverse transcription of mRNA to first strand cDNA. It is more difficult to obtain full length double strand cDNA than to obtain full length first, single strand cDNA. Our novel technique uses only first strand cDNA as the PCR template, so that the longest first strand cDNA could be synthesized by using reverse transcriptase without RNase H activity. The second problem overcome by our approach is that it is difficult to know the exact length of a cDNA insert in a cDNA library until the clone has been separated and it is difficult to know how many clones are needed to get a clone with full length. Our technique provides a mechanism by which the cDNA band of correct size can be obtained on the first pass or the amplification of cDNA ends can be repeated until the correct size of cDNA is obtained.

Another advantage of our method is the special designation of PCR primers. The amplification of cDNA toward the ends, which is contrary to normal gene structure, decreases the possibility of contamination in cDNA cloning from genomic DNA. This technique can also be used as a better alternative to existing methods for 5′ end primer extension because of its ability to specifically amplify cDNA ends using a graded series of amplification steps.

An important application of this approach is the analysis of the regulatory area of UTRs of mRNA's. As disclosed herein, no IRE structure was identified mRNAs designated as iron responsive indicating that gene expression can be influenced by other than iron responsive elements or mRNAs.

The following examples serve to illustrate the invention and are not intended to limit the invention in any way. It is expected that refinements of each step may be achieved with various reagents and protocols identified through routine experimentation these are intended to be within the scope of the invention.

### EXAMPLE 1

Iron is known to regulate the expression of genes that contain an iron responsive element (IRE) in their mRNA. However, iron-binding sites have been reported on genomic DNA (Dancis, A., Roman, D. G., Anderson, G. J., Hinnebusch, A. G., and Klausner, R. D. (1992) Proc. Natl. Acad. Sci. USA 89:3869-3873; Henle, E. S., Han, Z., Tang, N., Rai, P., Luo, Y., and Linn, S. (1999) J. Biol. Chem. 274:962-971; Neilanda, J. B. (1995) J. Biol. Chem. 270:26723-26726) and proteins functionally related to iron metabolism have been found in cell nuclei (Garre, C., Bianchi-Scarra, G., Sirito, M., Musso, M., and Ravazzolo, R. (1992) J. Cell Physiol. 153:477-482; Cai, C. X., Birk, D. E., and Linsenmayer, T. F. (1997) J. Biol. Chem 272:13831-12839). This suggests the possibility that iron may directly regulate expression of genes that do not have an IRE. We have identified a number of known genes that were not known to be iron responsive and number of novel genes that respond to cellular iron status (Ye, Z., and Connor, J. R. (2000) Nucleic Acids Res. 28:1802-1807; Ye, Z., and Connor, J. R. (1999) Biochem. Biophys. Res. Commun. 264:709-813). Cloning the full length of these cDNAs was critical to determining whether or not an IRE was involved in the response of these genes to iron.

Seven iron responsive mRNAs from previous screenings and the mRNA for the iron regulatory protein (IRP-1) (Barany, F. (1985) Proc. Natl. Acad. Sci. USA 82:4202-4206) were selected from mRNA of human astrocytoma cells and human brain for full length cloning with our novel method. The mRNAs were chosen because at least a partial sequence for each of them has been published in GenBank so that we could compare the efficiency of our cloning method to published results.

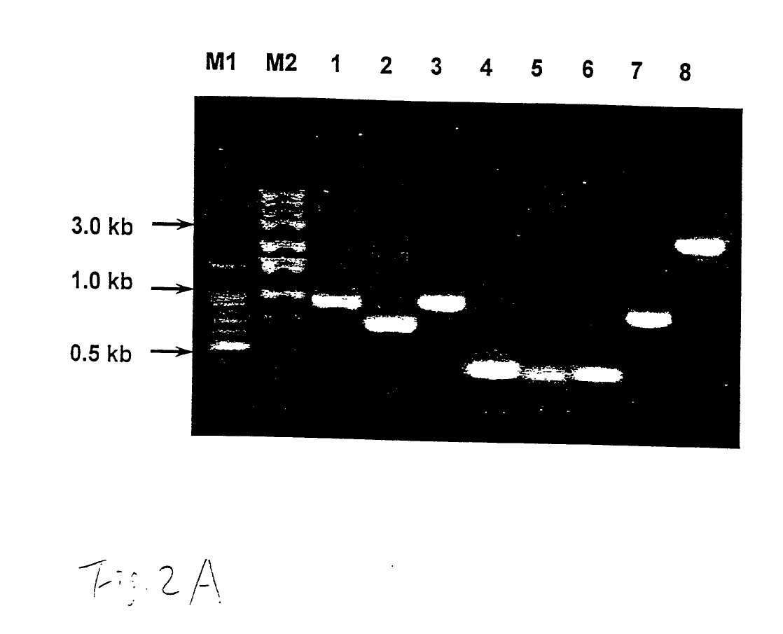

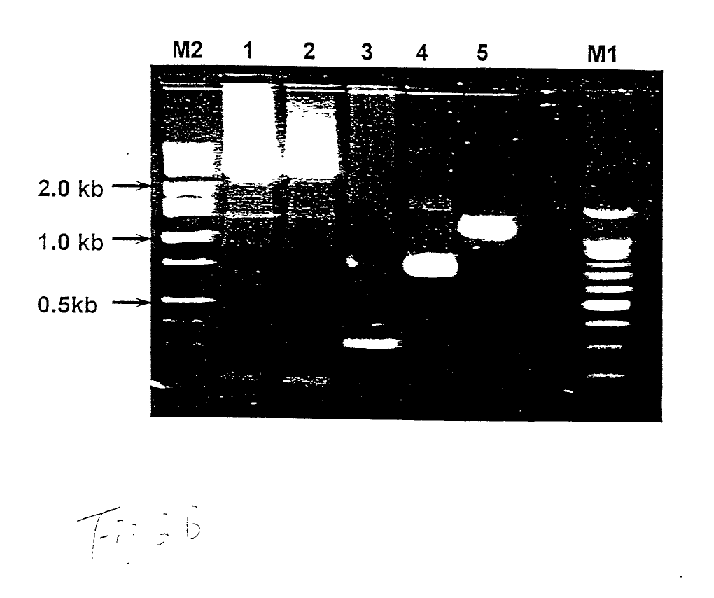

RNA reverse transcriptase without RNase H activity was used in the reverse transcription to obtain a single strand cDNA. The mRNA template was degraded with a mixture of RNase A and RNase H and the first strand cDNA was purified and the two ends ligated to form circular molecules. Two gene specific primers were designed from a segment of known sequence obtained in our previous study (Ye, Z., and Connor, J. R. (2000) Nucleic Acids Res. 28:1802-1807; Ye, Z., and Connor, J. R. (1999) Biochem. Biophys. Res. Commun. 264:709-813) and the 3′ end of the primers was toward to the 5′ or 3′ end of cDNA. Touchdown PCR was used to amplify both cDNA ends in one reaction. The PCR reaction product was detected on an agarose gel and the specific DNA band was purified and inserted to plasmid vector for sequencing (FIG. 2A).

Using our method for cloning, we obtained longer sequences at the 5′ and/or 3′ end for three of the test mRNAs (GAPDH, NEMO and Iron-inhibited ABC transporter) than what had been reported in GenBank. One cDNA (TEXREB107) had the same length as reported in GenBank. The sequence of Thy-1 cDNA is longer than the reported sequence in GenBank but still incomplete compared to the size indicated by Northern blot analysis. Three of the cDNAs (IRP-1, Calpain large polypeptide L2 and NADH dehydrogenase 1 beta subcomplex 9) were incompletely cloned because the sequence we obtained was shorter than that reported in GenBank. In addition, there is a possibility that the iron inhibited ABC transporter is encoded by two highly homologous mRNAs because two bands were obtained on Northern blots.

In order to obtain the complete sequence for the four mRNAs that were partially cloned and the homologous mRNA of an iron-inhibited ABC transporter, a second touchdown PCR was performed using new primers designed according to the sequence information from first PCR amplification. The PCR products were analyzed as described above (FIG. 2b). The second PCR amplification resulted in longer sequences at both the 5′ end and the 3′ end for Thy-1 mRNA than reported in GenBank and the size of cDNA is consistent with the size indicated on the Northern blot. For the iron inhibited ABC transporter, the second PCR amplification resulted in a specifically amplified product that may represent the difference between two cDNAs corresponding to the two bands that are indicated on Northern blots. The second PCR amplification also resulted in a longer sequence at the 5′ end of IRP-1 cDNA. After the second amplification we obtained the same sequence for NADH dehydrogenase 1 beta subcomplex 9 as that reported in GenBank. From the second PCR amplification for Calpain large polypeptide L2 we obtained same sequence at the 5′ end and a longer sequence at the 3′ end (180bp) than that reported in GenBank.

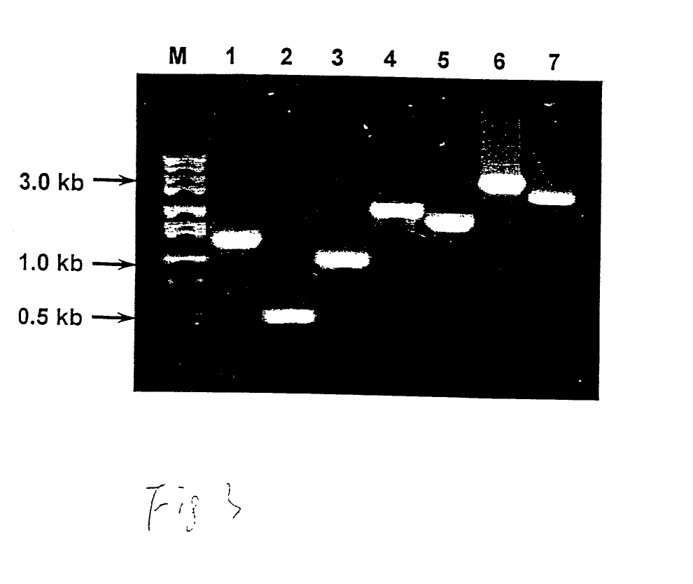

Because the first and second PCR amplifications used special templates (circularized first strand cDNAs) and a different primer designation (the 3′ end of primers are toward both ends of the cDNA) we used a third PCR to confirm that the cDNAs from first and second PCR runs are specific PCR products (FIG. 3). A third PCR amplification was also necessary because most of cDNAs cloned by our novel method contained new sequence data. One primer chosen against the novel sequence and the other primer from either a novel sequence or known sequence were used to amplify the specified cDNA sequence from the linear first strand cDNA. The PCR reactions on all seven of the cDNAs produced DNA that corresponded to the size that was predicted with the sequence information obtained from first and second PCR reactions (FIG. 3). The DNA from the third PCR reaction was sequenced and the sequence information was the same as that deduced from the first two PCR reactions.

In GenBank a sequence for NEMO and mRNA (see Table 1) has been reported, but our technique results in a sequence that is 74 bp longer. The additional 74 bp that we sequenced for NEMO mRNA have been previously reported on the glucose-6-phosphate dehydrogenase gene (G6PDH, GenBank number X55448.1). The G6PDH gene is in close proximity to the locus of NEMO gene on chromosome Xq28 (Jin, D. Y., Jeang K. T., J Biomed Sci, 6:115-20 (1999)). Our PCR and sequencing results prove these 74 bp belong to the first exon of the NEMO gene. The novel cDNA sequences for GAPDH and Thy-1 cloned by our method were also found on their respective genomic DNA (GenBank number J04038.1 and M11749). Thus, we confirmed the accuracy of our cloning method. Our results are compared to the sequences reported in GenBank in Table 1.

In addition to the sequence data, our study revealed two other novel observations. First, our results show that the Thy-1 mRNA may also encode another Thy-1 co-transcripted protein. Because the function of Thy-1 glycoprotein is still unclear but important in regulation of neuritic outgrowth and immune system activity, this new information may provide an important clue for discovering the function of Thy-1. Secondly, for the ABC transporter, two mRNAs were cloned and the sequence information revealed both of them contained the same open reading frame.

TABLE 1
Comparison of the mRNA Sequence Cloned by Our Novel
Method with the Sequence Published in Genbank

| | | Compared to | Compared to |
|---|---|---|---|
| Name | Genbank# of | GenBank | GenBank |
| and GenBank# | Compared | Sequence | Sequence |
| of Our Sequence | Sequence¹ | (5′ End)² | (3′ End)² |
| GAPDH mRNA | M 33197.1 | 43 bp Extension | No Difference |
| (GenBank# | | | |
| AF261085) | | | |
| Nemo mRNA | AF 091453 | 74 bp Extension | No Difference |
| (GenBank# | | | |
| AF261086) | | | |
| TEXREB107 | D 17554 | No Difference | No Difference |
| mRNA | | | |
| (GenBank# | | | |
| AF261087) | | | |
| IRP-1 mRNA | Z 11559 | 98 bp Extension | No Difference |
| (GenBank# | | | |
| AF261088) | | | |
| Calpain large | NM | No Difference | 180 bp Extension |
| polypeptide L2 | 001748.1 | | |
| mRNA | | | |
| (GenBank# | | | |
| AF261089) | | | |
| Thy-i mRNA | NM | 91 bp Extension | 588 bp Extension |
| (GenBank# | 006288.1 | | |
| AF261093) | | | |
| Iron inhibited ABC | | | Our sequence |
| transporter mRNA 1 | AJ005016.1 | 312 bp Extension | shorter (18 bp) |
| (GenBank# | | | |
| AF261092) | | | |
| Iron inhibited ABC | AJ005016.1 | 331 bp Extension | 993 bp Extension |
| transporter mRNA 2 | | | |
| (GenBank# | | | |
| AF261091) | | | |
| NADH | NM | No Difference | No Difference |
| dehydrogenase | 005005.1 | | |
| 1 beta subcomplex 9 | | | |
| mRNA (GenBank# | | | |
| 261090) | | | |

¹If there are several comparable sequences in Genbank, we choose the longest one for comparison. The area of poly-A tail were excluded from analysis.
²No difference is defined as less than 6 bp sequence difference between the compared sequences.

FIG. 1 is a schematic illustrating the principle of cDNA cloning by the methods of the invention. RNA reverse transcriptase without RNase H activity was used to synthesize the first strand cDNA. The mRNA template was degraded by RNases and the remaining first strand cDNA was purified and self-ligated to form circular molecules. Two gene specific primers (GSP 1 and GSP 2) were designed from a segment of known sequence obtained in a previous study (Ye, Z., and Connor, J. R. (2000) Nucleic Acids Res. 28:1802-1807; Ye, Z., and Connor, J. R. (1999) Biochem. Biophys. Res. Commun. 264:709-813). Both cDNA ends were amplified by a touchdown PCR reaction by using circularized first strand cDNAs as the template. The specifically amplified DNA was sequenced. To determine if the full length sequence of cDNA ends was obtained, the amplified DNA band was compared to the mRNA size predicted from Northern blot analysis and the sequence was compared to the sequences published in GenBank. If incomplete CDNA sequences were amplified in first PCR, another touchdown PCR could be applied by using circularized first strand cDNAs as templates and another pair of primers (GSP3 and GSP4) that were designed from the sequence information from the first PCR. The novel sequences were confirmed by a third PCR using linear first strand cDNA as a template. One primer of the third PCR was synthesized against the novel sequence (P1) and another PCR primer was from known sequencer novel sequence (P2). The specified amplifications from first and second PCR were confirmed if the size and sequence from a third PCR were consistent with the data from first and second PCR reaction.

FIG. 2A depicts the first PCR amplification to determine the size of the selected gene products visualized using ethidium bromide. The products were analyzed on 1% agarose gel. M1 and M2 are DNA molecular weight markers; 1, GAPDH; 2, NADH dehydrogenase 1 beta subcomplex 9; 3, DNA-binding Protein, TAXREB107; 4, NEMO Protein; 5, IRP-1; 6, calpain large polypeptide L2; 7, Thy-1; 8, iron-inhibited ABC transporter. The calculated sizes for GAPDH, NEMO were longer than that reported in GenBank and the size of the DNA binding protein TAXREB107 was similar to that reported in GenBank.

FIG. 2B depicts a second PCR amplification using new primers was performed on those genes whose size did not correspond to the size indicated by Northern blot analysis or to the size reported in GenBank. Lane 1, IRP-1; lane 2, calpain, large Polypeptide L2; lane 3, NADH dehydrogenase (ubiquinone) 1; lane 4, Thy-1; lane 5, iron-inhibited ABC transporter. Using the second set of primers, we obtained calculated lengths longer than that reported in GenBank for all five of the cDNAs examined. (M2 and Ml are DNA molecular weight markers.)

FIG. 3 depicts PCR amplification of cDNAs to confirm the novel cDNA sequences. The products of the PCR reaction were analyzed on 1% agarose gel. M is the DNA molecular weight Marker. Lane 1, GAPDH; lane 2, NEMO; lane 3, IRP-1; lane 4, calpain large polypeptide L2; lane 5, Thy-1; lane 6, ABC transporter (small band); lane 7. ABC transporter (large band). The sequences obtained by this amplification step correspond to the sequences obtained in the previous two PCR amplifications confirming that our cloning method is accurate.

### REFERENCES

1. Flouriot, G., Brand, H., and Gannon, F. (1999) Nucleic Acids Res. 27:e8

2. Loh, E. Y., Elliott, J. F., Cwirla, S., Lanier, L. L., and Davis, M. M. (1989) Science 243:217-220

3. Morinaga, N., Moss, J., and Vaughan, M. (1997) Proc.

Natl. Acad. Sci. USA 94:12926-12931

4. Dancis, A., Roman, D. G., Anderson, G. J., Hinnebusch, A. G., and Klausner, R. D. (1992) Proc. Natl. Acad. Sci. USA 89:3869-3873

5. Henle, E. S., Han, Z., Tang, N., Rai, P., Luo, Y., and Linn, S. (1999) J. Biol. Chem. 274:962-971

6. Neilanda, J. B. (1995) J. Biol. Chem. 270:26723-26726

7. Garre, C., Bianchi-Scarra, G., Sirito, M., Musso, M., and Ravazzolo, R. (1992) J. Cell Physiol. 153:477-482

8. Cai, C. X., Birk, D. E., and Linsenmayer, T. F. (1997) J. Biol. Chem 272:13831-12839

9. Ye, Z., and Connor, J. R. (2000) Nucleic Acids Res. 28:1802-1807

10. Ye, Z., and Connor, J. . (1999) Biochem. Biophys. Res. Commun. 264:709-813

11. Barany, F. (1985) Proc. Natl. Acad. Sci. USA 82:4202-4206

12. H. Hirling, et al., Nucleic Acids Res 20, 33-9 (1992).

13. Jin, D. Y., Jeang K. T., J Biomed Sci, 6:115-20 (1999).

What is claimed is:

1. A method for amplifying a polynucleotide sequence comprising:
obtaining a linear, single strand polynucleotide sample;
ligating the ends of said sample to form a circular shaped sample;
introducing first and second sequence specific primers to said circular sample; and
initiating a primer extension amplification reaction to increase copy number of said circular sample.

2. The method of claim 1 wherein said step of obtaining a linear, single strand nucleic acid sample further comprises the steps of:
obtaining a sample of mRNA;
contacting said mRNA with reverse transcriptase without RNase H so that a first strand cDNA-mRNA complex is formed, and degrading said mRNA to form a polynucleotide sample.

3. The method of claim 1 wherein said primer extension amplification reaction is a polymerase chain reaction.

4. The method of claim 1 wherein said polymerase chain reaction is employed with Taq polymerase or other heat-resisted DNA polymerase.

5. The method of claim 1 wherein said PCR is touchdown PCR.

6. The method of claim 2 further comprising the step of: harvesting said amplified nucleotide product.

7. The method of claim 1 wherein said ligase is T4 DNA ligase.

8. The method of claim 1 wherein said primer is a degenerate primer.

9. The method of claim 1 wherein said first and second primers are designed to hybridize to from about 4 to about 35 contiguous bases from a sequence known or suspected to be present in said nucleic acid sample.

10. The method of claim 1 wherein said first primer comprises a 3′ end of the same which is toward the 5′ end of the nucleic acid sample.

11. The method of claim 1 wherein one of said primers comprises a 3′ end of the same which is toward the 3′ end of said nucleic acid sample.

12. A method for amplifying a nucleic acid molecule including the 5′ and 3′ ends comprising:
circularizing said nucleic acid molecule;
contacting said nucleic acid with first and second primers; and
introducing a polymerase and a supply of nucleotide bases to said circularized nucleic acid molecule so that an amplification reaction occurs; wherein said region of said nucleic acid molecule outside of said first and second primers including the 3′ and 5′ ends of said molecule is amplified.

13. The method of claim 1 wherein said ligase is T4 DNA ligase.

14. The method of claim 1 wherein said primer is a degenerate primer.

15. The method of claim 1 wherein said forward and reverse primers are designed to hybridize to from about 4 to about 35 contiguous bases from a sequence known or suspected to be present in said nucleic acid sample.

16. The method of claim 1 wherein said one of said primers comprises a 3′ end of the same which is toward the 5′ end of the nucleic acid sample.

17. The method of claim 1 wherein one of said primers comprises a 3′ end of the same which is toward the 3′ end of said nucleic acid sample.

18. A method of cloning a full length cDNA sequence from an mRNA sample comprising: obtaining a sample of mRNA;
transcribing said mRNA to cDNA in th e absence of RNase H activity;
degrading said mRNA so that a single strand of cDNA is obtained;
ligating the ends of said cDNA;
selecting forward and reverse gene specific primers from known sequence of a gene suspected to be present in said cDNA; and
amplifying said cDNA by an extension chain reaction.

19. A method of sequencing a full length coding DNA or mRNA for a gene comprising;
obtaining a sample of mRNA;
transcribing said mRNA to cDNA in the absence of RNase H activity;
degrading said mRNA so that a single strand of cDNA is obtained;
ligating the ends of said cDNA;
selecting forward and reverse gene specific primers from known sequence of a gene suspected to be present in said cDNA;
amplifying said cDNA by a polymerase chain reaction; to obtain an amplified product and thereafter;
inserting said amplified product into a vector for sequencing.

20. A set of nucleotide primers for use in PCR amplification of circularized cDNA comprising:
a forward primer of from about 4 to about 35 contiguous bases capable of hybridizing to a gene which is to be amplified, and
a reverse primer of from about 4 to about 35 contiguous bases capable of hybridizing to a gene which is to be amplified, wherein said forward primer is towards the 3′ end of said gene and said reverse primer is towards the 5′ end of said gene.

21. A kit for amplifying first strand cDNA from a sample of mRNA comprising:
a DNA ligase,
a DNA polymerase,
a reverse transcriptase without RNase H activity;
an enzyme for degrading mRNA from a cDNA-mRNA hybrid;
each of the four deoxynucleoside triphosphates (dATP, dCTP, dGTP, and dTTP.

22. A full length CDNA sequence said sequence determined by the method of claim 17.

23. A cloned nucleic acid obtained by the method of claim 1.

24. A method for amplifying a nucleic acid sequence comprising:
obtaining a linear, single strand nucleic acid sample;
ligating the ends of said sample to form a circular shaped sample;
introducing first and second sequence specific primers to said circular sample; wherein said sequence specific primers each have a 3′ end directed toward the 5′ or 3′ end of said specific sequence, and
initiating an amplification reaction to amplify said circular sample.

25. A method for amplifying a nucleic acid sequence comprising:
obtaining a linear, single strand nucleic acid sample;
ligating the ends of said sample to form a circular shaped sample;
introducing first and second sequence specific primers to said circular sample; wherein said sequence specific primers each have a 3′ end directed toward the 5′ or 3′ end of said specific sequence, and
initiating a polymerase chain amplification reaction to amplify said circular sample.
